(19) United States
(12) Patent Application Publication
(10) Pub. No.: US 2025/0006352 A1
(43) Pub. Date: Jan. 2, 2025

(54) INTERFACE FOR ROUTING DATA TRANSMISSIONS IN RESPONSE TO DYNAMIC QUEUING OF SERVICE REQUESTS

(71) Applicant: American Well Corporation, Boston, MA (US)

(72) Inventor: Roy Schoenberg, Boston, MA (US)

(21) Appl. No.: 18/759,374

(22) Filed: Jun. 28, 2024

Related U.S. Application Data
(60) Provisional application No. 63/524,602, filed on Jun. 30, 2023.

(51) Int. Cl. G16H 40/20; G16H 10/60; G16H 80/00; H04L 9/40

(52) U.S. Cl. CPC G16H 40/20; G16H 10/60; G16H 80/00; H04L 63/08

(57) ABSTRACT

Systems and methods including an interface for routing data transmissions in response to dynamic queuing of service requests. A data processing system includes the interface in which one or more medical service providers within structured data records, such as electronic health records (EHR) or electronic medical records (EMR), can indicate that they are available to provide services to one or more patients. When a medical service provider indicates that he or she is available to provide additional medical services, one or more patients who have requested a corresponding medical service can be automatically routed to that medical service provider.

### CLAIM OF PRIORITY

This application claims priority under 35 U.S.C. § 119 (c) to U.S. Patent Application Ser. No. 63/524,602, filed on Jun. 30, 2023, the entire contents of which are hereby incorporated by reference.

### TECHNICAL FIELD

This disclosure generally relates to systems and methods including an interface for routing requests over a network. Specifically, this disclosure relates to an interface for establishing a wireless communication with a remote device and a client device based on data describing a type of the request.

### BACKGROUND

In electronic health record (EHR) systems, medical service providers, such as physicians, are generally segmented depending on in which healthcare provider network they are located. A physician can log into an EHR system and see records for one or more patients within that system. Often, the medical service provider cannot access patients outside of the network in which that medical service provider is located. In addition, one or more patients may be awaiting service within the respective networks in which those patients are located.

### SUMMARY

This specification describes systems and methods including an interface for routing data transmissions in response to dynamic queuing of service requests. A data processing system includes the interface in which one or more medical service providers within structured data records, such as electronic health records (EHR) or electronic medical records (EMR), can indicate that they are available to provide services to one or more patients. When a medical service provider indicates that he or she is available to provide additional medical services, one or more patients who have requested a corresponding medical service can be automatically routed to that medical service provider. The one or more patients can be included in a common pool of patients that form a queue. In this example, each queue is created for patients requesting a similar service, for example, a similar consultation with a specific type of physician. The data processing system is configured to accept requests from many various different kinds of client devices and systems with different data requirements, translate these requests into a common request format, and query the EHR system for available medical service providers. The medical service providers can indicate whether they are available to provide the service by specifying what types of services they provide. For a given medical service provider, or multiple medical service providers, a queue of patients can form in which the next available patient is allocated to the first available medical service provider automatically.

The interface included in this system solves one or more of the following technical problems. Generally, the service requests are sent from multiple different devices which may be associated with different protocols and/or formats. For example, the interface is configured to work with many different request types that are themselves configured to interface with different application programming interfaces (APIs). The interface is configured to process any of these requests of varying formats. The Interface is configured for parsing the requests for relevant features, such as what type of medical services being requested, a location of the medical service, or other relevant information for the service request that may indicate which medical service provider is the most appropriate for that particular request. The data processing system then generates a request that is configured to route the patient into the medical service providers particular EHR system. Specifically, the interface is configured to generate a request with the requisite format, protocol, and so forth so that the medical service provider can be connected to the patient.

This system enables any medical service provider in any EHR system to receive requests from any patient. For example, a medical service provider can opportunistically take a patient that is not in that medical service providers EHR system without requiring that physician to interact with a programming system that is outside of that physician's EHR system. The interface is configured to access data within the physician's EHR system automatically to respond to the requests.

Each of the following embodiments are configured to enable one or more of the foregoing advantages.

In a general aspect, a method implemented by a data processing system includes determining, at a provider gateway system, that a particular provider is authorized for one or more digital communication sessions, wherein the particular provider is represented in the provider gateway system by a digital identifier; responsive to determining authorization, rendering, in a provider graphical user interface for that digital identifier, a control, selection of which transmits a request to register the particular provider with a queue processing system; receiving, by the provider gateway system through the provider graphical user interface, selection data specifying selection of the control, with the selection data specifying the digital identifier; and responsive to receipt of the selection data, automatically: retrieving, from a hardware storage device, one or more data records associated with the digital identifier; based on the retrieved one or more data records, instructing the queue processing system to register the particular provider with the queue processing system by: transmitting to the queue processing system the digital identifier and an indication that the particular provider represented by the digital identifier is authorized for the one or more digital communication sessions, wherein the queue processing system maintains queues with entries representing users requesting digital communication sessions; receiving, from the queue processing system, an indication of a user for a digital communication session with the particular provider, with the indication of the user specifying another digital identifier; registering the user with the provider gateway system by storing, in a hardware storage device, a data structure representing the user, with the data structure structured with data specifying the digital identifier of the user; and by the provider gateway system, rendering another provider graphical user interface for the digital identifier of the provider that displays a visual representation of a console and a visual representation of the user within the console.

In some implementations, retrieving the one or more data records associated with the digital identifier comprises accessing a computing system associated with the digital identifier, the computing system hosting the one or more data records through an application interface that is different from another application interface associated with one or more other data records associated with a different computing system associated with another digital identifier.

In some implementations, retrieving the one or more data records associated with the digital identifier comprises accessing authorization data associated with the digital identifier.

In some implementations, the process includes accessing, using the authorization data, a computing system hosting the one or more data records.

In some implementations, the queues with entries representing users requesting digital communication sessions are each associated with a particular type of medical service provided by providers associated with the queues.

In a general aspect, a process implemented by a data processing system includes causing rendering, in a graphical user interface of an electronic health record system, a control, selection of which transmits to a data processing system a request to establish a communication channel between the electronic health record system and a client device, with the request including an identifier of a provider of the electronic health record system; responsive to selection data specifying selection of the control, accessing, in memory, one or more data structures representing one or more users requesting a consultation, with each data structure structured with one or more fields storing data representing one or more characteristics of a user; parsing, by a parser of the data processing system, fields in the data structures to identify a given data structure with data representing one or more characteristics of a user eligible for a consultation with the provider; generating, by the data processing system, a medical record number for a user represented by the given data structure; registering, by the data processing system, the user with the electronic health record system by: causing storage, in memory of the electronic health record system, of a data structure representing the user, with the data structure structured with data representing the medical record number; and causing updating of the graphical user interface with: one or more visual representations specifying the registered user, with the one or more visual representations juxtaposed in the graphical user interface to one or more electronic health record icons; and one or more controls juxtaposed to the one or more electronic health record icons, selection of at least one of the one or more controls causing launching of a communication channel between the electronic health record system and the client device.

In some implementations, accessing, in memory, the one or more data structures comprises accessing a computing system associated with a digital identifier, the computing system hosting the one or more data records through an application interface that is different from another application interface associated with one or more other data records associated with a different computing system associated with another digital identifier.

In some implementations, accessing, in memory, the one or more data structures comprises accessing authorization data associated with the user; and accessing, using the authorization data, a computing system hosting the one or more data records.

In some implementations, the data representing one or more characteristics of a user eligible for a consultation with the provider comprises a representation of a type of medical service.

In a general aspect, the processes described herein are performed by a data processing system, comprising at least one processor; and at memory storing instructions that, when executed by the at least one processor, cause the at least one processor to perform the operations described herein.

In a general aspect, one or more non-transitory computer readable media store instructions for execution by at least one processor to perform the operations described herein.

These and other aspects, features, and implementations can be expressed as methods, apparatus, systems, components, program products, means or steps for performing a function, and in other ways. These and other aspects, features, and implementations will become apparent from the following descriptions, including the claims.

### BRIEF DESCRIPTION OF THE DRAWINGS

FIG. 1 shows a data processing system.

FIG. 2 shows data flow for processing image data and generating EHR request data.

FIG. 3A shows an example application interface.

FIG. 3B shows an example application interface.

FIG. 3C shows an example user interface.

FIG. 4 shows an example user interface.

FIG. 5 shows an example user interface.

FIG. 6 shows an example user interface.

FIG. 7 shows an example user interface.

FIG. 8 shows an example flow diagram.

FIG. 9 shows an example flow diagram.

FIG. 10 is a block diagram of an example computing system.

### DETAILED DESCRIPTION

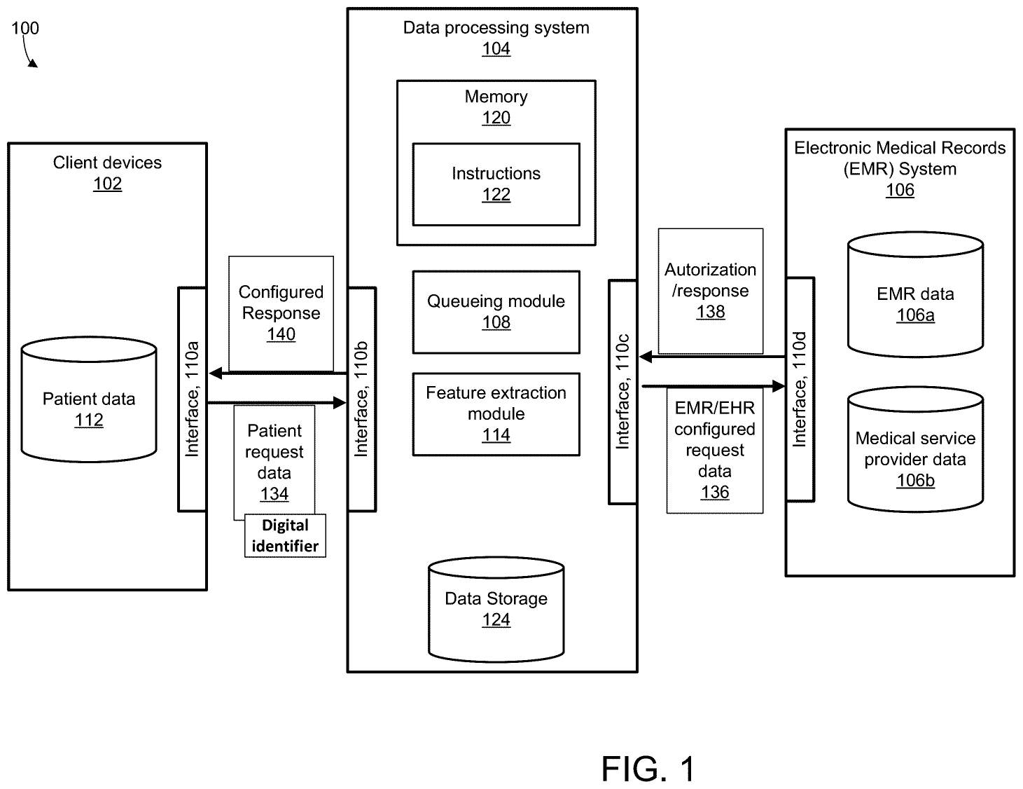

FIG. 1 shows an environment 100 for a data processing system 104. The data processing system 104 is configured to determine which medical patients should be routed to communicate with respective medical service providers. Specifically, the data processing system 104. specifically, the data processing system 104 includes an interface 110, shown as interfaces 110a-d, that is configured to identify medical service providers who are authorized to provide perspective digital communication sessions with patients based on requests submitted from client device 102 associated with a patient. The data processing system 104 is configured to receive patient request data 134 from a client device 102. The data processing system 104 receives the patient request data 134 through an interface 110 (e.g., a provider gateway system). The data processing system 104 processes the patient request data 134 and generates A corresponding request 136 that is compatible with the target EMR system 106. The request 136 is configured to establish a virtual communication session between the medical service provider and the patient. The virtual communication session is established once a response 138 identifying a particular medical service provider or indicating that the medical service provider is available, is sent through the interface 110 back to the client device 102 as a configured response 140 that is compatible with the client device 102. Once the virtual communication session handshake has been completed, the client device may communicate with the medical service provider through the interface 110, such as using video conferencing, as subsequently described.

The data processing system 104 is configured to extract features from the patient request data 134, configure a request 136 for EMR systems, and send the request 136 to the EMR system 106 through the interface 110. Generally, the patient request data includes 134 data describing the patient from patient data 112 and also includes a request for specific medical service. For example, the medical service can include a type of medical service provider requested, such as a primary care physician, an ophthalmologist, a neurologist, an car, nose, and throat (ENT) physician, a gastroenterologist, or any other medical service provider. These features are extracted from the patient request data 134 by the data processing system 104, and a corresponding request 136 is generated to request service from a medical service provider that is responsive to the request data 134. The data processing system configures the request so that the target EMR system 106 can process and receive the request and so that the request can reach as many medical service providers as possible.

The EMR system 106 receives the request 136 and sends a response 138 indicating which medical service providers are eligible to be responsive to the request 134. In some implementations, the EMR system includes the data processing system 104. The EMR system 106 can identify which medical service providers are eligible to respond to the request 136 based on input received from the medical service providers. In some implementations, the EMR system 106 receives in the configured request data 136 a specific medical service provider who is responsive to the request 134 from the patient. In this example, the response 138 includes a list of eligible medical service providers. The data processing system 104 then queues the requests receive from the patient to the eligible medical service providers.

The data processing system 104 is configured to accept patient request data 134 from many kinds of client devices 102 for different EMR systems. These requests are generally configured for APIs that are specific to different EMR systems. The different requests can therefore have specific protocols and or formats from one another that are not all compatible with every EMR system 106. The data processing system 104 is configured to process the request data 134 regardless of the format or protocol being used. For example, the data processing system 104 can receive request data 134 configured for dozens of different APIs associated with respective EMR systems. The data processing system 104 is configured to extract relevant features from these requests 134 and configure request data 136 that is compatible for the particular EMR system 106 associated with the eligible medical service provider. The request data 134 are associated with an identifier, such as an index, that uniquely identifies the particular patient.

The requests from different patients can be combined into a common queueing interface, as subsequently described. The interface, or provider gateway, enables a connection between computing systems or networks that would otherwise be incompatible for medical service providers to access as a common pool of patients for providing medical services. The interface enables a medical service provider can therefore access a patient who is waiting for a service, regardless of what computing system is associated with that patient's data. The interface routes patient request data 134 to the medical service providers from multiple computing networks or systems. The interface improves an efficiency of processing the patient requests for medical services that would otherwise be prevented because of incompatible communication between the medical service provider systems and the corresponding patient systems or networks hosting the patient data. The interface ensures that there are no patients who are stuck waiting for medical services for a long time because of a backlog associated with a particular computing system or network associated with that patient. In some implementations, accessing the patient data from a specific computing system associated with the patient can include providing authentication to access that patient data. The queuing system described herein is configured to access the authentication information for accessing patient data to preserve patient privacy and maintain data security while also accessing data from different computing systems associated with different patients. The automated authentication provides flexibility for queuing patients while also maintaining data security. The patient can provide the authentication criteria when registering with the queuing interface.

The patient request data 134 can include one or more features of patient data 112 that are stored at the client device is 102. The interface 110 of the data processing system 104 receives the data and is configured to parse the patient request data 134 to extract the features using the feature extraction module 114. The extracted features of the patient request data 134 are used for configuring a request 136 for the EMR system 106. For example, the features may include data such as a location of the patient, a type of medical service being requested, a type of medical service provider being requested, one or more demographics of the patient, a time of day, a current medical service provider associated with the patient, any data entered by the patient that describes the request, or other features that are relevant to the request. Generally, the patient request data 134 are configured to satisfy the one or more requirements of an API that is associated with a particular EMR system associated with the portal, system, application, and so forth through which the patient is requesting the medical service. The various interfaces through which the patient may submit their request data 134 are not affected by the data processing system 104 processing the request. In other words, the data processing system 104 is invisible to the end user at the client device 102. The client simply requests a medical service provider as they would normally, and the data processing system 104 intercepts the request, processes the request, and selects a medical service provider, and sends an appropriately configured request to the respective EMR system 106 associated with that medical service provider.

The data processing system 104 is configured to send the request 136 using queuing that is configured using the queuing module 108. The queuing module 108 of the data processing system 104 is configured to determine which specific medical service provider of the EMR system 106 should receive the request for medical services that is in the configured request data 136.

The data processing system 104 is configured to receive authorization from medical service providers to be selected by the queuing module 108. In an example, the data processing system functions as a provider gateway system. In this example, the data processing system 104 is configured to determine that a particular medical service provider is authorized for one or more digital communication sessions, wherein the particular provider is represented in the provider gateway system by a digital identifier. Once authorization 138 is determined, the data processing system is configured to enable a medical service provider device to render, for a digital identifier identifying the patient, a control, selection of which transmits a request to register the particular provider with a queue processing system. The control can include a button or other activation mechanism on the user interface of the device of the medical service provider. The authorization is sent in data 138 and allows that particular medical service provider to be selected by the queuing module as an eligible medical service provider for responding to a request from the client device 102.

The data processing system 104 is configured to receive, through the provider graphical user interface, selection data specifying selection of the control, with the selection data specifying the digital identifier that identifies the patient. The data processing system 104, responsive to receipt of the selection data, automatically: retrieving, from a hardware storage device, one or more data records associated with the digital identifier from EMR system 106. The EMR system 106 includes EMR data 106a and medical service provider data 106b. These data 106a-b can be sent to the data processing system 104 for storage and data storage 124 once authorization has been granted.

Based on the retrieved one or more data records, The data processing system 104 instructs the queue processing system (e.g., querying module 108) to register the particular provider with the queue processing system by transmitting to the queue processing system the digital identifier and an indication that the particular provider represented by the digital identifier is authorized for the one or more digital communication sessions. The queue processing system maintains queues with entries representing users requesting digital communication sessions. The data processing system 104 receives from the queue processing system 108 an indication of a user for a digital communication session with the particular provider, with the indication of the user specifying another digital identifier. The data processing system 104 is configured to register the user with the provider gateway system by storing, in a hardware storage device 124, a data structure representing the user, with the data structure structured with data specifying the digital identifier of the user.

The data processing system 104 renders another provider graphical user interface for the digital identifier of the provider that displays a visual representation of a console and a visual representation of the user within the console. The data processing system 104 establishes a communication session that uses each of these representations. In some implementations, their communication session can include video conferencing between a single medical service provider and a single patient, a single patient and multiple medical service providers, and so forth. In some implementations, the communication session includes an audio communication session between a single patient and a single medical service provider, or a single patient and multiple medical service providers. In some implementations, the communication session is a video conferencing session in which any of the participants may interact with a user interface to communicate with any other participant in the video conferencing session.

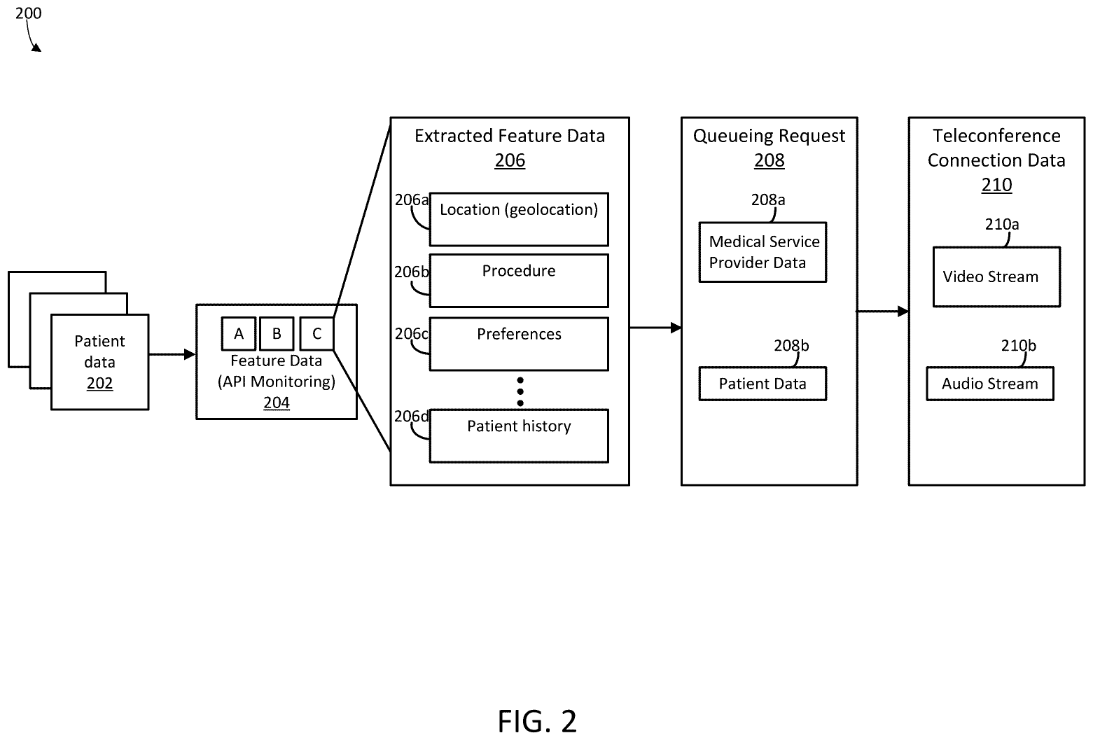

FIG. 2 shows data flow 200 for processing image data and generating HER/EMR request data 136 of FIG. 1. In some implementations, the data flow 200 occurs within the feature extraction module 114 of FIG. 1. The data processing system is configured to receive patient data 202, which can be included in the patient request data 134 previously described. The feature extraction module 114 is configured to extract one or more features 204 from the patient data 202. For example, the feature extraction module 114 can extract extracted feature data 206 such as a location 206a, a procedure type 206b, patient preferences 206c, patient history 206d, or any other such data that are relevant to the request for a medical service by the patient.

The data processing system 104 uses the extracted features 206 to match a patient to a respective medical service provider that has provided authorization to the data processing system through an interface, as described previously. Each medical service provider that provides authorization is eligible to receive patient requests that are assigned by the queuing module 108. However, not every medical service provider is eligible for every patient request. The feature extraction module 114, by performing the workflow 200, determines which particular medical service providers should be eligible for particular request. In this way, neither the physician nor the patient needs to specify a specific corresponding entity for a next communication session. Rather, a patient can simply include various data that specify what type of medical service provider the patient is seeking, and the physician can simply state that he or she is eligible to receive requests while providing data regarding what requests that physician can receive. The data processing system 104 can receive patient data requests of any data type and or format and translating that request into one in which any eligible physician can be responsive. This bridge is a technical gap between various EMR systems 106 and patient portals that use different API's and other technologies for configuring requests to respective EMR systems 106. Patients are not restricted based on the type of portal they are using or which specific EMR system they may be sending a request, but instead are eligible to receive service from any eligible medical service provider.

The data processing system 104, once the features 206 are extracted, is configured to generate a queuing request 208. The queuing request 208 specifies what types of medical service providers 208a are eligible to respond to the particular request and also includes patient data 208b that are relevant to the medical service provider for responding to the request. This ensures that any patient can access any eligible medical service provider, regardless of the particulars of the computing systems, portals, networks, APIs, or other system details specific to the patient or medical service provider. In this way, any patient can be routed to a specific medical service providers EHR desktop to which the medical service provider may be restricted. Correspondingly, in this way any patient can reach a medical service provider who is eligible to respond to the request of the patient, regardless of the portal being used by the patient.

The data processing system is configured to enable data connections between patients associated with different networks or systems that may otherwise be incompatible with each other. For example, patients may be associated with different EHR databases storing data in different systems that may not be interconnected or commonly accessible without the interface described herein. Specifically, the queues allow data to be pulled from these various different systems and pooled into a common system so that a medical service provider 208 can respond to the next patient in the queue regardless of the particular system associated with that patient. For example, different patients in different networks can be accessible by the medical service provider 208. This allows the medical service provider 208 to access patients outside of the network in which that medical service provider is located. One or more patients may be awaiting service within the respective networks in which those patients are located, and a medical service provider who is available can contact that patient to provide faster service.

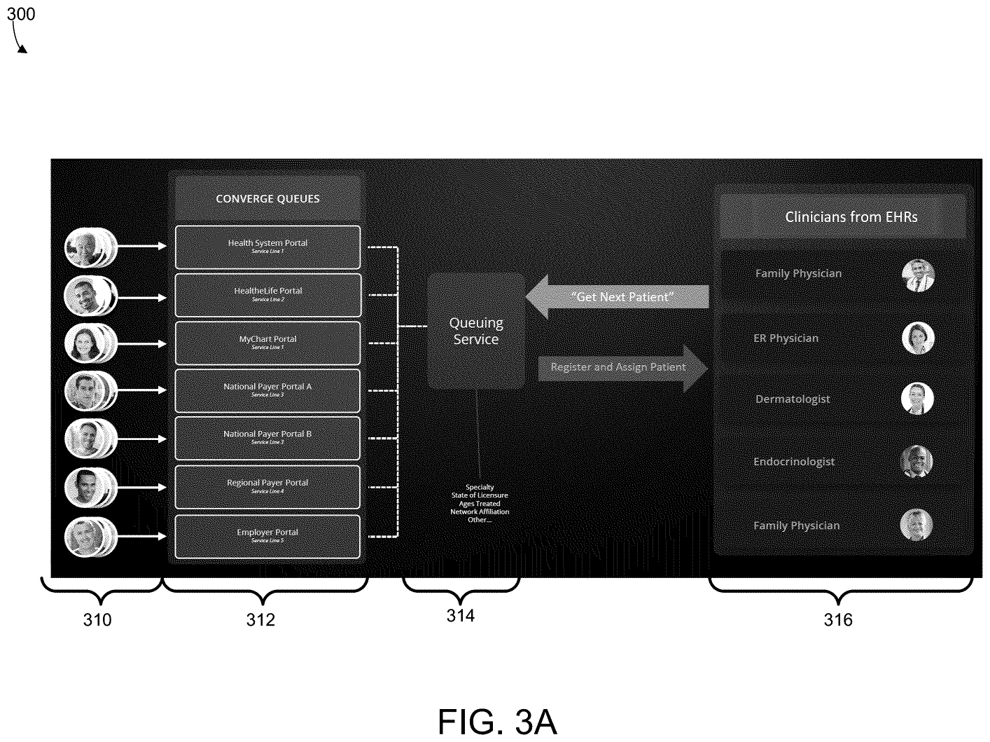

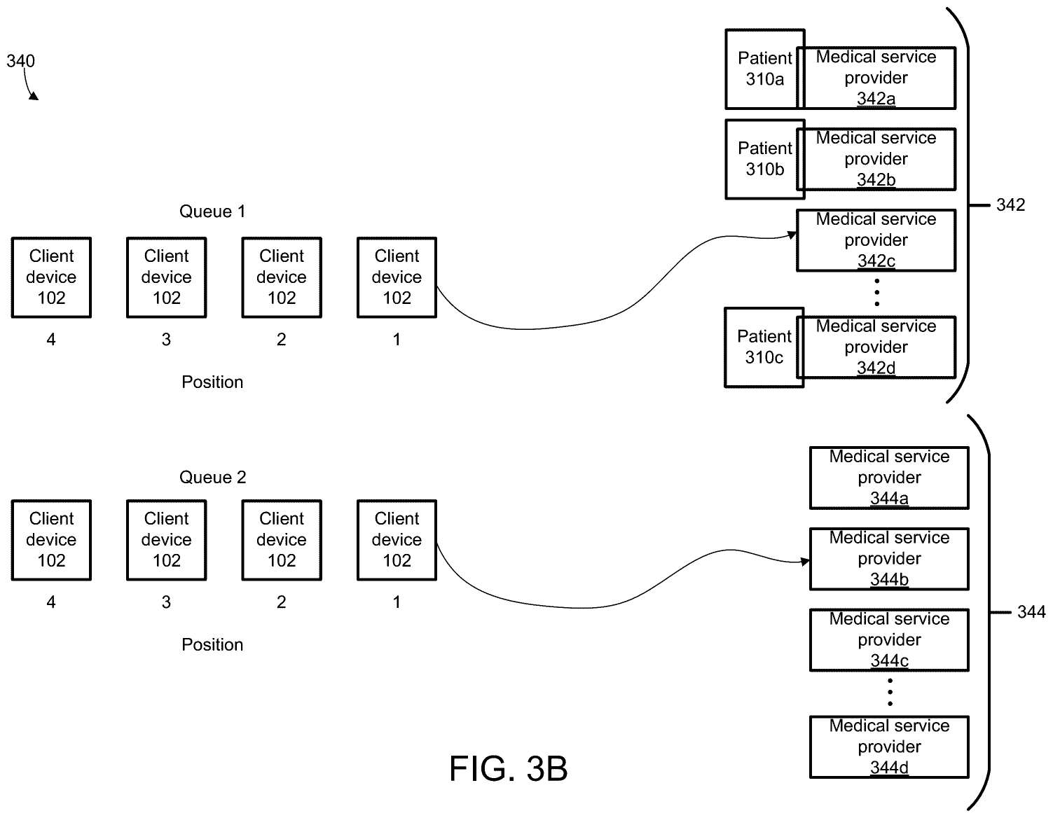

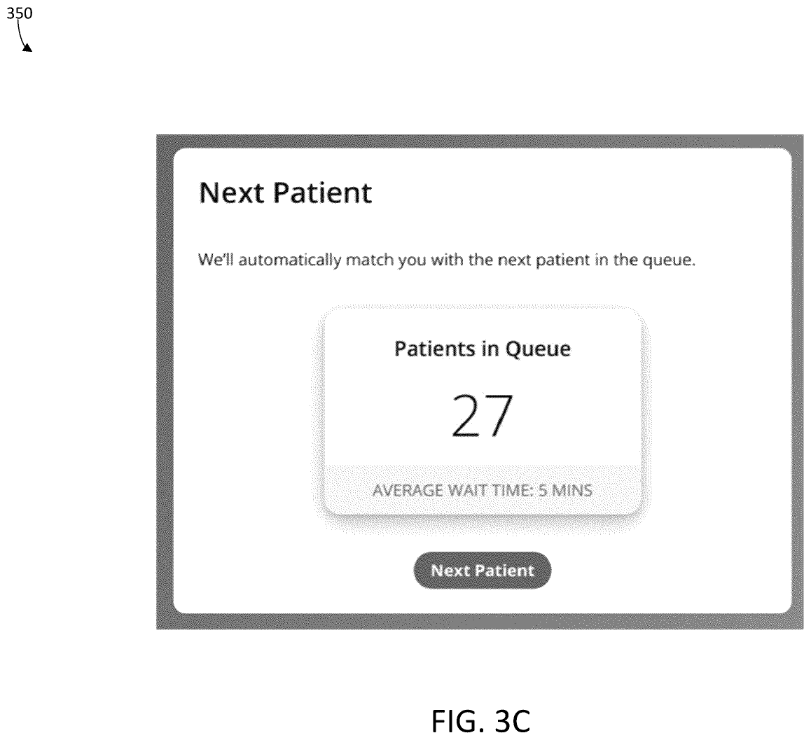

FIG. 3A shows an example system 300 including a queuing service. The queuing service 314 (part of queuing module 108 of FIG. 1) is shown within the system 300. Various patients 310 can submit to various queues 312 in the system 300. Each of these queues 312 is associated with a specific interface on a client device of the respective patients 310. For example, a first patient may be related to a health system portal which is a first service, a second patient may be associated with a health life portal interface, a third patient may be associated with a “my chart” portal, a fourth patient may be associated with a national payer portal type A, a fifth patient may be associated with a national payer portal type B, a sixth patient may be associated with a regional payer portal, and a seventh patient may be associated with an employer portal. While seven example patients are shown, there can be many more different types of portals, interfaces, APIs, or other access points for accessing respective medical service providers. The queuing service 314 is configured to receive data that includes extracted features from each of the requests from the patients 310. The queuing service can therefore combine all the patients 310 into a single set of queues that are specific to types of clinicians or medical service providers that are being requested by the respective patients 310. In some implementations, a single queue can form for all the patients 310. In some implementations, multiple queues are formed based on one or more features in their requests, such as location, type of medical service provider required, and so forth.

The queuing service 314 is configured to register the patients with various EMR systems 316 of respective medical service providers. The querying service 314 is configured to access all these different EMR systems 316 regardless of their specific formats or protocols required to access these EMR systems 316. Once a particular medical service provider becomes available, a response is sent out that requests a next patient from the queuing service 314. The response can include their response 138, described previously with respect to FIG. 1. Once a new patient is requested, the queuing service 314 selects the next patient 310 in the queue and sends the patient to the appropriate eligible medical service provider who requested the patient. Neither the patient nor the physician needs to activate the service to initiate the next communication session. Rather, the system 300 is configured to automatically match the next available patient with the next available physician so that a communication session can be initiated automatically.

In some implementations, a medical service provider 316, who participates in this process by the EHR, may elect to pick a “next patient” from either an interface inside the EHR or can branch out from the EHR into a converge interface and review/pick a next patient(s) from there. The provider can therefore interact with the queue using either interface or a combination thereof.

FIG. 3B shows an example queuing system 340. In the system 340, multiple queues are generated depending on one or more features that are common to the client devices associated with patients in the queue. For example, all the patients in queue 1 may be requesting a same type of medical service for which specific pool 342 of medical service providers 342a-d are eligible. In this example, the patients of queue 2 may also have features that are common to one another, but distinct from the patients of the first queue. These patients may be requesting medical service providers 344a-d from a different pool of medical service providers 344 than those being requested by the patients of the first queue. In this example, the queues 1, 2 and operate in parallel and operate for respective different pools of medical service providers 342, 344 from one another.

FIG. 3C shows an example user interface 350. The user interface shows what a medical service provider might see in his or her respective EMR user interface. For example, the medical service provider is shown that there are 27 patients in queues for which that medical service provider is eligible to respond. In this example, there is a single queue of 27 patients, and the average wait time for patients is about 5 minutes. Therefore, a physician can determine about how many different patients he or she can see from the list of eligible patients in the near future. The length of the queue of patients may decrease as other eligible medical service providers are paired with respective patients. When the medical service provider selects the next patient control 352 in the user interface, the medical service provider is automatically paired with the next available patient in the queue. As previously described, a queuing module 108 determines which medical service providers are eligible to see which patients and generates a queue for medical service providers that are eligible to respond to the respective patient requests.

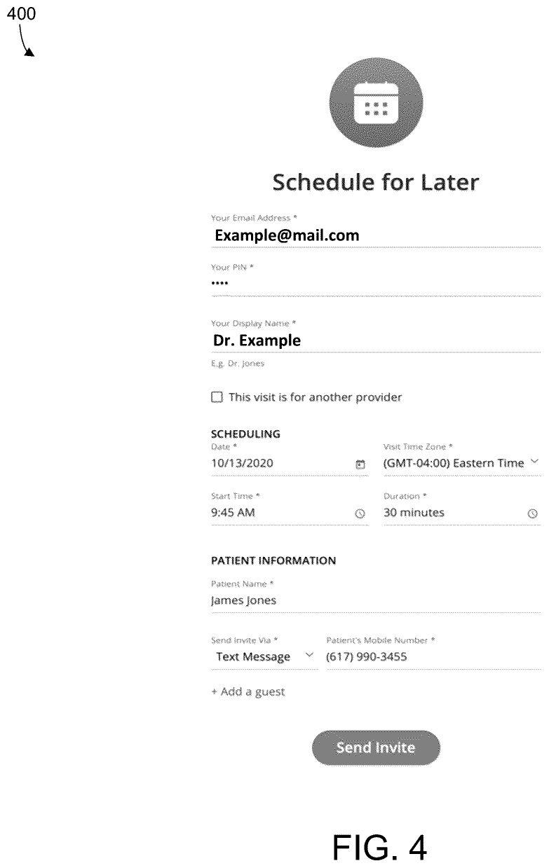

FIG. 4 shows an example user interface 400 for registering a medical service provider with the queuing module 108. in this example, a medical service provider can indicate when he or she is available to host a communication session for an eligible patient in the queue. Rather than immediately being paired with the corresponding patient, the medical service provider can schedule the communication session for a later time. However, the medical service provider is paired with a corresponding patient for which the medical service provider is eligible to provide a service based on the request received from the patient.

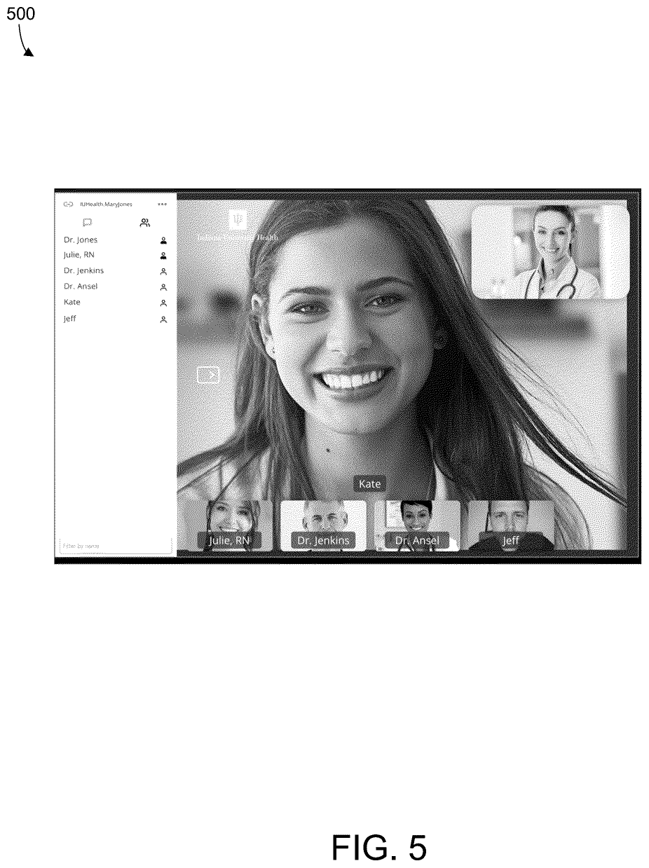

FIG. 5 shows an example user interface 500. The user interface 500 can include a video conferencing interface through which a communication session can occur. Each of the patient and the medical service provider can view the interface 500. In some implementations, the interface 500 for a communication session is a video conferencing interface. The interface 500 can be automatically generated to connect the patient to the medical service provider once the queuing process has been completed. The interface 500 enables communication between the patient and the corresponding medical service provider regardless of the network or computing system associated with the patient. In some implementations, patient data specific to the patient can be presented along with communications with the patient to the medical service provider. The patient data can be automatically accessed from the computing system associated with the patient, such as a specific EHR system.

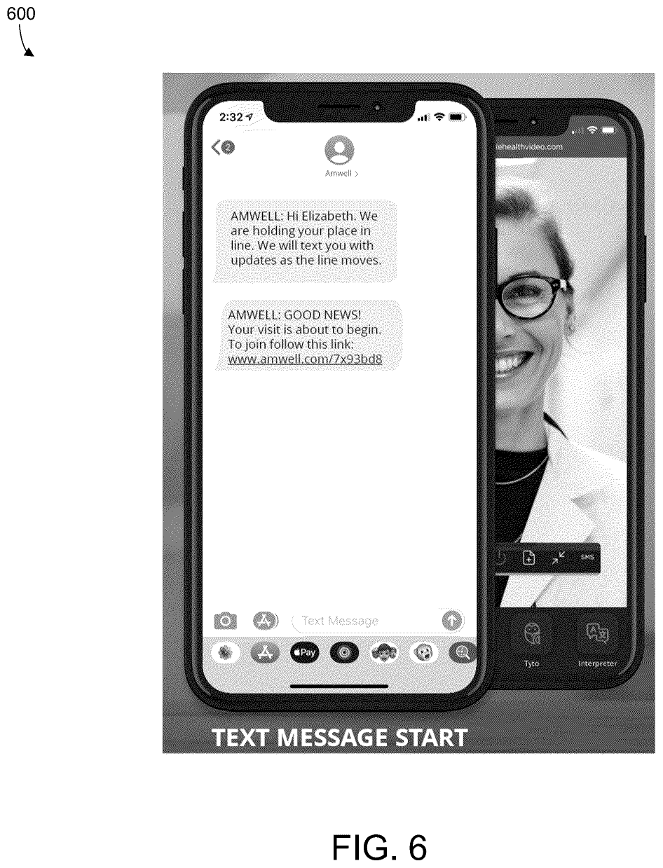

FIG. 6 shows an example user interface 600. The interface 600 is a text-based interface in which a user receives a response from the medical service provider or from the data processing system 104 as a text-based message. For example, the text-based message can be sent as an SMS message through the interface 600. The message in the interface 600 can indicate to a patient where in the queue the patient currently is, what an expected wait time might be, and when the medical service provider becomes available. When the medical service provider becomes available, the interface 600 can indicate to the patient how to access the communication session, such as the interface 500 described with respect to FIG. 5. For example, the text-based message in the interface 600 can provide a hyperlink or other link directly to the interface 500.

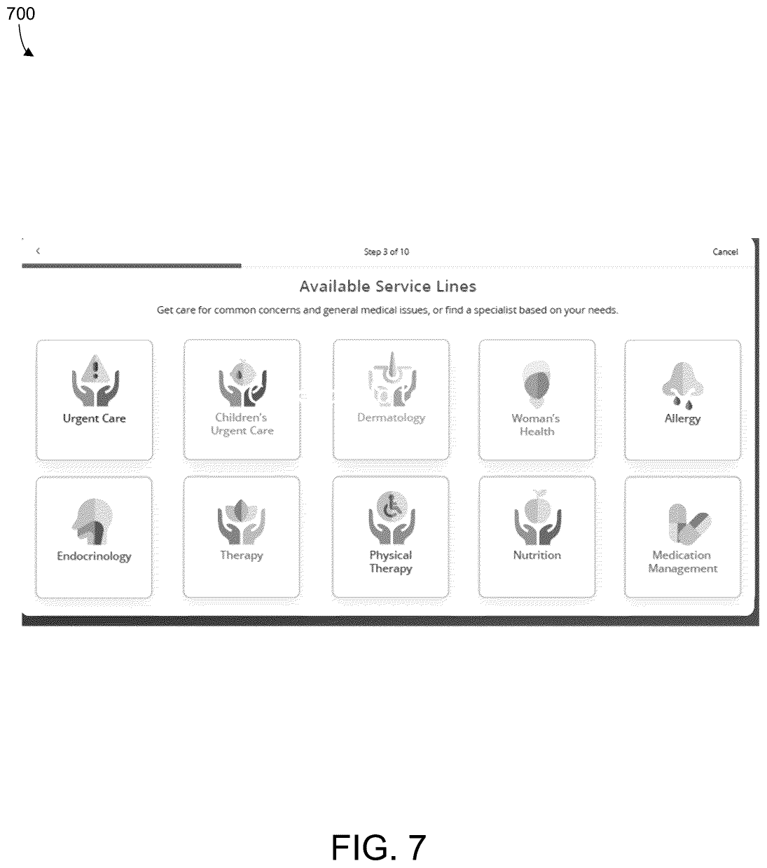

FIG. 7 shows an example user interface 700. The interface 700 can be presented to a patient to help the patient configure the request 134 that is sent to the data processing system. For example, the user interface can specify which service lines are available to the patient through the interface 110. a patient can be assigned to a particular queue based on, for example, a type of service selected by the patient. In the example interface 700 of FIG. 7, the patient can select a service from a list including urgent care, children's urgent care, dermatology, woman's health, allergies, endocrinology, physical therapy, mental therapy, nutrition, or medication management. These examples are illustrative rather than exhaustive. In an example, each of the types of medical services shown in the interface 700 can cause the patient to be routed to a specific queue that is for a set of medical service providers as described in reference to FIG. 3B. in some implementations, a patient is set to a same queue regardless of the type of medical service selected, but the medical service type is used to determine which medical service provider is eligible to respond to the patient request. In this example, all patients are in a common queue, but only specific medical service providers are eligible for specific patients in the queue. In another example, each medical service provider can be associated with a particular queue of eligible patients for that medical service provider.

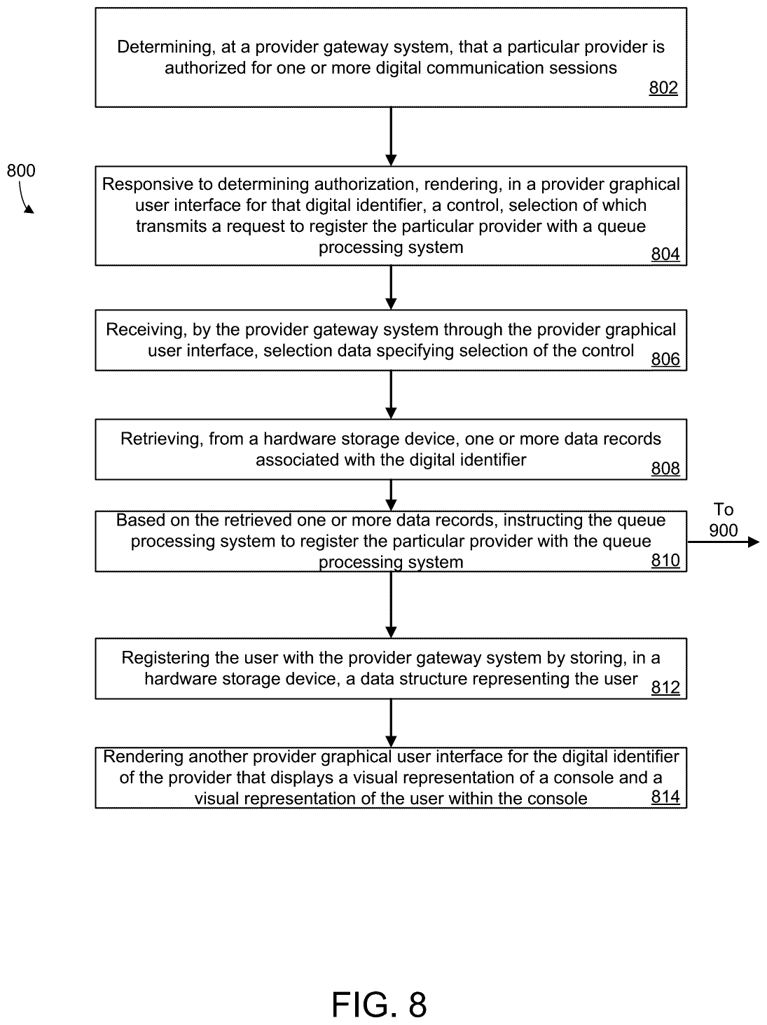

FIG. 8 shows a flow diagram showing a process 800 for routing data transmissions in response to dynamic queuing of service requests. The process 800 includes determining (802), at a provider gateway system, that a particular provider is authorized for one or more digital communication sessions, wherein the provider is represented in the provider gateway system by a digital identifier. The process 800 includes, responsive to determining authorization, rendering (804), in a provider graphical user interface for that digital identifier, a control, selection of which transmits a request to register the provider with a queue processing system. The process 800 includes receiving (806), by the provider gateway system through the provider graphical user interface, selection data specifying selection of the control, with the selection data specifying the digital identifier. The process 800 includes, responsive to receipt of the selection data, automatically retrieving (808), from a hardware storage device, one or more data records associated with the digital identifier. The process 800 includes, based on the retrieved one or more data records, instructing (810) the queue processing system to register the provider with the queue processing system. The process 800 includes automatically registering (812) the user with the provider gateway system by storing, in a hardware storage device, a data structure representing the user, with the data structure structured with data specifying the digital identifier of the user. The process 800 includes, by the provider gateway system, rendering (814) another provider graphical user interface for the digital identifier of the provider that displays a visual representation of a console and a visual representation of the user within the console.

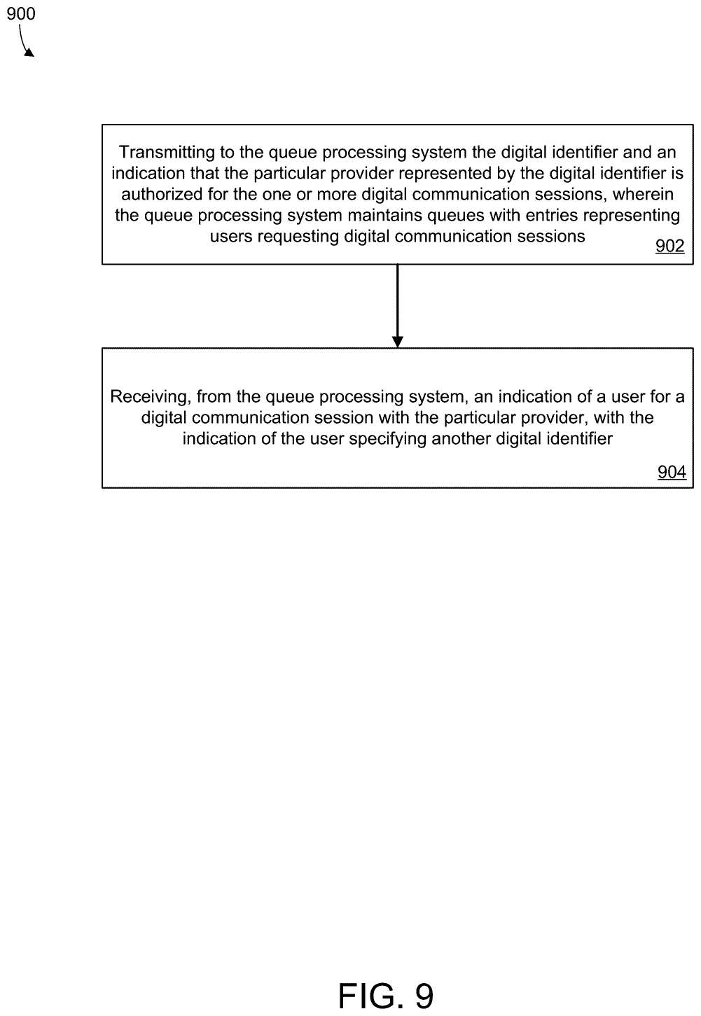

FIG. 9 shows a flow diagram showing a process 900 for routing data transmissions in response to dynamic queuing of service requests. The process 900 includes, based on the retrieved one or more data records, instructing the queue processing system to register the provider with the queue processing system by transmitting (902) to the queue processing system the digital identifier and an indication that the provider represented by the digital identifier is authorized for the one or more digital communication sessions. The queue processing system maintains queues with entries representing users requesting digital communication sessions. The process 900 includes receiving, from the queue processing system, an indication of a user for a digital communication session with the provider, with the indication of the user specifying another digital identifier.

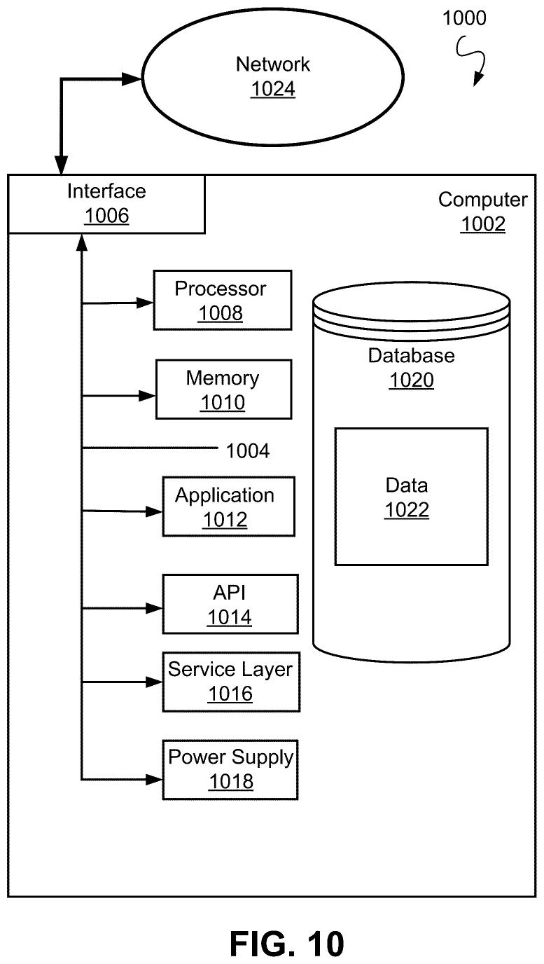

FIG. 10 is a block diagram of an example computing system 1000 used to provide computational functionalities associated with described algorithms, methods, functions, processes, flows, and procedures described in the present disclosure, according to some implementations of the present disclosure. The illustrated computer 1002 is intended to encompass any computing device such as a server, a desktop computer, a laptop/notebook computer, a wireless data port, a smart phone, a personal data assistant (PDA), a tablet computing device, or one or more processors within these devices, including physical instances, virtual instances, or both. The computer 1002 can include input devices such as keypads, keyboards, and touch screens that can accept user information. Also, the computer 1002 can include output devices that can convey information associated with the operation of the computer 1002. The information can include digital data, visual data, audio information, or a combination of information. The information can be presented in a graphical user interface (UI) (or GUI).

The computer 1002 can serve in a role as a client, a network component, a server, a database, a persistency, or components of a computer system for performing the subject matter described in the present disclosure. The illustrated computer 1002 is communicably coupled with a network 1024. In some implementations, one or more components of the computer 1002 can be configured to operate within different environments, including cloud-computing-based environments, local environments, global environments, and combinations of environments.

At a high level, the computer 1002 is an electronic computing device operable to receive, transmit, process, store, and manage data and information associated with the described subject matter. According to some implementations, the computer 1002 can also include, or be communicably coupled with, an application server, an email server, a web server, a caching server, a streaming data server, or a combination of servers.

The computer 1002 can receive requests over network 1024 from a client application (for example, executing on another computer 1002). The computer 1002 can respond to the received requests by processing the received requests using software applications. Requests can also be sent to the computer 1002 from internal users (for example, from a command console), external (or third) parties, automated applications, entities, individuals, systems, and computers.

Each of the components of the computer 1002 can communicate using a system bus 1004. In some implementations, any or all the components of the computer 1002, including hardware or software components, can interface with each other or the interface 1006 (or a combination of both), over the system bus 1004. Interfaces can use an application programming interface (API) 1014, a service layer 1016, or a combination of the API 1014 and service layer 1016. The API 1014 can include specifications for routines, data structures, and object classes. The API 1014 can be either computer-language independent or dependent. The API 1014 can refer to a complete interface, a single function, or a set of APIs.

The service layer 1016 can provide software services to the computer 1002 and other components (whether illustrated or not) that are communicably coupled to the computer 1002. The functionality of the computer 1002 can be accessible for all service consumers using this service layer. Software services, such as those provided by the service layer 1016, can provide reusable, defined functionalities through a defined interface. For example, the interface can be software written in JAVA, C++, or a language providing data in extensible markup language (XML) format. While illustrated as an integrated component of the computer 1002, in alternative implementations, the API 1014 or the service layer 1016 can be stand-alone components in relation to other components of the computer 1002 and other components communicably coupled to the computer 1002. Moreover, any or all parts of the API 1014 or the service layer 1016 can be implemented as child or sub-modules of another software module, enterprise application, or hardware module without departing from the scope of the present disclosure.

The computer 1002 includes an interface 1006. Although illustrated as a single interface 1006 in FIG. 10, two or more interfaces 1006 can be used according to needs, desires, or particular implementations of the computer 1002 and the described functionality. The interface 1006 can be used by the computer 1002 for communicating with other systems that are connected to the network 1024 (whether illustrated or not) in a distributed environment. Generally, the interface 1006 can include, or be implemented using, logic encoded in software or hardware (or a combination of software and hardware) operable to communicate with the network 1024. More specifically, the interface 1006 can include software supporting one or more communication protocols associated with communications. As such, the network 1024 or the hardware of the interface can be operable to communicate physical signals within and outside of the illustrated computer 1002.

The computer 1002 includes a processor 1008. Although illustrated as a single processor 1008 in FIG. 10, two or more processors 1008 can be used according to particular implementations of the computer 1002 and the described functionality. Generally, the processor 1008 can execute instructions and can manipulate data to perform the operations of the computer 1002, including operations using algorithms, methods, functions, processes, flows, and procedures as described in the present disclosure.

The computer 1002 also includes a database 1020 that can hold data (for example, data 1022) for the computer 1002 and other components connected to the network 1024 (whether illustrated or not). For example, database 1020 can be an in-memory, conventional, or a database storing data consistent with the present disclosure. In some implementations, database 1020 can be a combination of two or more different database types (for example, hybrid in-memory and conventional databases) according to particular implementations of the computer 1002 and the described functionality. Although illustrated as a single database 1020 in FIG. 10, two or more databases (of the same, different, or combination of types) can be used according to particular implementations of the computer 1002 and the described functionality. While database 1020 is illustrated as an internal component of the computer 1002, in alternative implementations, database 1020 can be external to the computer 1002.

The computer 1002 also includes a memory 1010 that can hold data for the computer 1002 or a combination of components connected to the network 1024 (whether illustrated or not). Memory 1010 can store any data consistent with the present disclosure. In some implementations, memory 1010 can be a combination of two or more different types of memory (for example, a combination of semiconductor and magnetic storage) according to implementations of the computer 1002 and the described functionality. Although illustrated as a single memory 1010 in FIG. 10, two or more memories 1010 (of the same, different, or combination of types) can be used according to implementations of the computer 1002 and the described functionality. While memory 1010 is illustrated as an internal component of the computer 1002, in alternative implementations, memory 1010 can be external to the computer 1002.

The application 1012 can be an algorithmic software engine providing functionality according to implementations of the computer 1002 and the described functionality. For example, application 1012 can serve as one or more components, modules, or applications. Further, although illustrated as a single application 1012, the application 1012 can be implemented as multiple applications 1012 on the computer 1002. In addition, although illustrated as internal to the computer 1002, in alternative implementations, the application 1012 can be external to the computer 1002.

The computer 1002 can also include a power supply 1018. The power supply 1018 can include a rechargeable or non-rechargeable battery that can be configured to be either user- or non-user-replaceable. In some implementations, the power supply 1018 can include power-conversion and management circuits, including recharging, standby, and power management functionalities. In some implementations, the power-supply 1018 can include a power plug to allow the computer 1002 to be plugged into a wall socket or a power source to, for example, power the computer 1002 or recharge a rechargeable battery.

There can be any number of computers 1002 associated with, or external to, a computer system containing computer 1002, with each computer 1002 communicating over network 1024. Further, the terms “client,” “user,” and other appropriate terminology can be used interchangeably, as appropriate, without departing from the scope of the present disclosure. Moreover, the present disclosure contemplates that many users can use one computer 1002 and one user can use multiple computers 1002.

Implementations of the subject matter and the functional operations described in this specification can be implemented in digital electronic circuitry, in tangibly embodied computer software or firmware, in computer hardware, including the structures disclosed in this specification and their structural equivalents, or in combinations of one or more of them. Software implementations of the described subject matter can be implemented as one or more computer programs. Each computer program can include one or more modules of computer program instructions encoded on a tangible, non-transitory, computer-readable computer-storage medium for execution by, or to control the operation of, data processing apparatus. Alternatively, or additionally, the program instructions can be encoded in/on an artificially generated propagated signal. The example, the signal can be a machine-generated electrical, optical, or electromagnetic signal that is generated to encode information for transmission to suitable receiver apparatus for execution by a data processing apparatus. The computer-storage medium can be a machine-readable storage device, a machine-readable storage substrate, a random or serial access memory device, or a combination of computer-storage mediums.

Computer readable media (transitory or non-transitory, as appropriate) suitable for storing computer program instructions and data can include all forms of permanent/non-permanent and volatile/non-volatile memory, media, and memory devices. Computer readable media can include, for example, semiconductor memory devices such as random-access memory (RAM), read only memory (ROM), phase change memory (PRAM), static random-access memory (SRAM), dynamic random-access memory (DRAM), erasable programmable read-only memory (EPROM), electrically erasable programmable read-only memory (EEPROM), and flash memory devices. Computer readable media can also include, for example, magnetic devices such as tape, cartridges, cassettes, and internal/removable disks. Computer readable media can also include magneto optical disks and optical memory devices and technologies including, for example, digital video disc (DVD), CD ROM, DVD+/−R, DVD-RAM, DVD-ROM, HD-DVD, and BLURAY. The memory can store various objects or data, including caches, classes, frameworks, applications, modules, backup data, jobs, web pages, web page templates, data structures, database tables, repositories, and dynamic information. Types of objects and data stored in memory can include parameters, variables, algorithms, instructions, rules, constraints, and references. Additionally, the memory can include logs, policies, security or access data, and reporting files. The processor and the memory can be supplemented by, or incorporated in, special purpose logic circuitry.

Any claimed implementation is applicable to at least a computer-implemented method; a non-transitory, computer-readable medium storing computer-readable instructions to perform the computer-implemented method; and a computer system comprising a computer memory interoperable coupled with a hardware processor configured to perform the computer-implemented method or the instructions stored on the non-transitory, computer-readable medium.

While this specification contains many specific implementation details, these should not be construed as limitations on the scope of what may be claimed, but rather as descriptions of features that may be specific to implementations. Certain features that are described in this specification in the context of separate implementations can also be implemented, in combination, in a single implementation. Conversely, various features that are described in the context of a single implementation can also be implemented in multiple implementations, separately, or in any suitable sub-combination. Moreover, although previously described features may be described as acting in certain combinations and even initially claimed as such, one or more features from a claimed combination can, in some cases, be excised from the combination, and the claimed combination may be directed to a sub-combination or variation of a sub-combination.

A number of embodiments have been described. Nevertheless, it will be understood that various modifications may be made without departing from the scope of the embodiments herein. Accordingly, other embodiments are within the scope of the following claims.

What is claimed is:

1. A method implemented by a data processing system, comprising:
determining, at a provider gateway system, that a particular provider is authorized for one or more digital communication sessions, wherein the particular provider is represented in the provider gateway system by a digital identifier;
responsive to determining authorization, rendering, in a provider graphical user interface for that digital identifier, a control, selection of which transmits a request to register the particular provider with a queue processing system;
receiving, by the provider gateway system through the provider graphical user interface, selection data specifying selection of the control, with the selection data specifying the digital identifier; and
responsive to receipt of the selection data, automatically:
retrieving, from a hardware storage device, one or more data records associated with the digital identifier;
based on the retrieved one or more data records, instructing the queue processing system to register the particular provider with the queue processing system by:
transmitting to the queue processing system the digital identifier and an indication that the particular provider represented by the digital identifier is authorized for the one or more digital communication sessions, wherein the queue processing system maintains queues with entries representing users requesting digital communication sessions;
receiving, from the queue processing system, an indication of a user for a digital communication session with the particular provider, with the indication of the user specifying another digital identifier;
registering the user with the provider gateway system by storing, in a hardware storage device, a data structure representing the user, with the data structure structured with data specifying the digital identifier of the user; and
by the provider gateway system, rendering another provider graphical user interface for the digital identifier of the provider that displays a visual representation of a console and a visual representation of the user within the console.

2. The method of claim 1, wherein retrieving the one or more data records associated with the digital identifier comprises accessing a computing system associated with the digital identifier, the computing system hosting the one or more data records through an application interface that is different from another application interface associated with one or more other data records associated with a different computing system associated with another digital identifier.

3. The method of claim 1, wherein retrieving the one or more data records associated with the digital identifier comprises accessing authorization data associated with the digital identifier; and
accessing, using the authorization data, a computing system hosting the one or more data records.

4. The method of claim 1, wherein the queues with entries representing users requesting digital communication sessions are each associated with a particular type of medical service provided by providers associated with the queues.

5. A method implemented by a data processing system, comprising:
causing rendering, in a graphical user interface of an electronic health record system, a control, selection of which transmits to a data processing system a request to establish a communication channel between the electronic health record system and a client device, with the request including an identifier of a provider of the electronic health record system;
responsive to selection data specifying selection of the control, accessing, in memory, one or more data structures representing one or more users requesting a consultation, with each data structure structured with one or more fields storing data representing one or more characteristics of a user;
parsing, by a parser of the data processing system, fields in the data structures to identify a given data structure with data representing one or more characteristics of a user eligible for a consultation with the provider;
generating, by the data processing system, a medical record number for a user represented by the given data structure;
registering, by the data processing system, the user with the electronic health record system by:
causing storage, in memory of the electronic health record system, of a data structure representing the user, with the data structure structured with data representing the medical record number; and
causing updating of the graphical user interface with:
one or more visual representations specifying the registered user, with the one or more visual representations juxtaposed in the graphical user interface to one or more electronic health record icons; and
one or more controls juxtaposed to the one or more electronic health record icons, selection of at least one of the one or more controls causing launching of a communication channel between the electronic health record system and the client device.

6. The method of claim 5, wherein accessing, in memory, the one or more data structures comprises accessing a computing system associated with a digital identifier, the computing system hosting the one or more data records through an application interface that is different from another application interface associated with one or more other data records associated with a different computing system associated with another digital identifier.

7. The method of claim 5, wherein accessing, in memory, the one or more data structures comprises accessing authorization data associated with the user; and
accessing, using the authorization data, a computing system hosting the one or more data records.

8. The method of claim 5, wherein the data representing one or more characteristics of a user eligible for a consultation with the provider comprises a representation of a type of medical service.

9. A data processing system, comprising:
at least one processor; and
at memory storing instructions that, when executed by the at least one processor, cause the at least one processor to perform operations comprising:
causing rendering, in a graphical user interface of an electronic health record system, a control, selection of which transmits to a data processing system a request to establish a communication channel between the electronic health record system and a client device, with the request including an identifier of a provider of the electronic health record system;
responsive to selection data specifying selection of the control, accessing, in memory, one or more data structures representing one or more users requesting a consultation, with each data structure structured with one or more fields storing data representing one or more characteristics of a user;
parsing, by a parser of the data processing system, fields in the data structures to identify a given data structure with data representing one or more characteristics of a user eligible for a consultation with the provider;
generating, by the data processing system, a medical record number for a user represented by the given data structure;
registering, by the data processing system, the user with the electronic health record system by:
causing storage, in memory of the electronic health record system, of a data structure representing the user, with the data structure structured with data representing the medical record number; and
causing updating of the graphical user interface with:
one or more visual representations specifying the registered user, with the one or more visual representations juxtaposed in the graphical user interface to one or more electronic health record icons; and
one or more controls juxtaposed to the one or more electronic health record icons, selection of at least one of the one or more controls causing launching of a communication channel between the electronic health record system and the client device.

10. The data processing system of claim 9, wherein accessing, in memory, the one or more data structures comprises accessing a computing system associated with a digital identifier, the computing system hosting the one or more data records through an application interface that is different from another application interface associated with one or more other data records associated with a different computing system associated with another digital identifier.

11. The data processing system of claim 9, wherein accessing, in memory, the one or more data structures comprises accessing authorization data associated with the user; and
accessing, using the authorization data, a computing system hosting the one or more data records.

12. The data processing system of claim 9, wherein the data representing one or more characteristics of a user eligible for a consultation with the provider comprises a representation of a type of medical service.
